(19) United States
(12) Patent Application Publication
(10) Pub. No.: US 2026/0202391 A1
(43) Pub. Date: Jul. 16, 2026

(54) METHOD FOR CONTINUOUSLY DETERMINING THE LACTOSE, PROTEIN, AND FAT CONTENT OF MILK

(71) Applicant: Endress+Hauser Flowtec AG, Reinach (CH)

(72) Inventors: Elena Kapussuzi, Freising (DE); Stefan Pflüger, München (DE); Anne Habermehl, München (DE); Ronen Hurni, Reinach (CH)

(21) Appl. No.: 19/137,033

(22) PCT Filed: Nov. 16, 2023
(86) PCT No.: PCT/EP2023/081999

§ 371 Date: Jun. 9, 2025

(30) Foreign Application Priority Data
Dec. 9, 2022 (DE) 10 2022 132 798.1

(51) Int. Cl. G01N 33/04; G01N 27/08; G01N 33/06

(52) U.S. Cl. CPC G01N 33/04; G01N 27/08; G01N 33/06

(57) ABSTRACT

A method for determining, in particular in a continuous manner, the lactose content a, protein content b, and fat content c of a flowable medium, in particular milk, a milk substitute, and/or a milk product, comprises the method steps of: —ascertaining the frequency-dependent relative permittivity of the medium over a frequency range of a frequency spectrum, wherein the frequency spectrum has an upper limit of 50 GHz, in particular 25 GHz, preferably 14 GHz, and the frequency spectrum has a lower limit of 10 MHz, in particular 50 MHz, preferably 85 MHz; and —calculating the lactose content a, protein content b, and fat content c on the basis of the ascertained relative permittivity and on the basis of a lactose reference, a protein reference, and a fat reference. Also disclosed is a measuring assembly.

The invention relates to a method for determining, in particular continuously, a lactose content a, protein content b and fat content c of a flowable medium, in particular milk, a milk substitute and/or a milk product, and to a measuring assembly for determining a lactose content a, protein content b and fat content c of a flowable medium, in particular milk, a milk substitute and/or a milk product.

It is possible, by means of microwaves, to determine the physical variables of permittivity and loss factor of a medium in a process line. From these two variables—measured either at one or over many different frequencies—it is possible to draw conclusions regarding application-specific parameters, for example the proportion of water in a mixture of water and other non-polar or weakly polar components or a solid content in a liquid medium.

The established transmission/reflection measurement is described in L. F. Chen, C. K. Ong, C. P. Neo, V. V. Varadan, V. K. Varadan—“Microwave Electronics, Measurement and Materials Characterization,” John Wiley & Sons Ltd., 2004. For this purpose, the microwave signal interfaces at two different positions at the medium in a container or measuring tube, the scatter parameters (transmission and optionally reflection) are measured between these interface structures, and the mentioned physical properties of the medium are calculated from the measured scatter parameters.

WO 2018 121927 A1 teaches a measuring assembly for analyzing properties of a flowing medium by means of microwaves. In addition to the microwave antennas, the measuring assembly has an electrically insulating lining layer on the inner peripheral surface of the measuring tube. This lining layer forms a dielectric waveguide via which at least part of the one microwave signal can travel from a first microwave antenna to a second microwave antenna. One application for such a measuring assembly is the determination of the proportions of solids in the liquid medium being conveyed. WO 2021/099152 A1 teaches a microwave antenna which has a front section in contact with the medium, via which the excitation signal is emitted into the medium.

Milk and any (intermediate) products obtained therefrom can be described as a mixture of different components, consisting mainly of water, milk fat and other solids, with the other solids substantially comprising proteins, carbohydrates (including in particular lactose) and, in small quantities, minerals.

During the processing chain from raw milk to the finished milk product, the proportions of these components are important parameters for controlling the processes using open-loop and closed-loop control, for process and quality control, and for balancing product flows. It is common practice to use standard methods to determine the proportions using laboratory samples. This means that only a few samples can be evaluated and that the analysis result is only available with a significant delay after the sampling process. Although process-oriented spectroscopic analysis in the infrared range with automated sampling is possible, this is firstly very cost-intensive and secondly only based on the small volume of samples taken at comparatively long time intervals. Such an analysis therefore has only limited suitability with regard to process control.

DE102017131269A1 discloses a method for continuously determining the fat content of milk with varying solids contents using a Coriolis flowmeter and a microwave sensor. These types of measuring devices can be integrated into milk processing processes. A disadvantage of the disclosed solution is that it is not possible to separate the solids in order to precisely determine the protein and lactose content.

The object of the invention is to remedy this.

The object is achieved by the method according to claim 1 and by the measuring assembly according to claim 13.

The method according to the invention for determining, in particular continuously, a lactose content a, protein content b and fat content c of a, in particular flowable, medium, in particular milk, a milk substitute and/or a milk product comprises the method steps of:
determining a frequency-dependent relative permittivity of the medium across a frequency range of a frequency spectrum,
   wherein the frequency spectrum has an upper limit of 50 GHz, in particular 25 GHz, and preferably 14 GHz,
   wherein the frequency spectrum has a lower limit of 10 MHz, in particular 50 MHz and preferably 85 MHz; and
calculating the lactose content a, protein content b and fat content c on the basis of the relative permittivity determined and on the basis of a lactose reference, a protein reference and a fat reference.

By providing the lactose, protein and fat reference and including them in the calculation of the individual components of the solid, the lactose contribution can be distinguished from the protein contribution, despite having a very similar density and permittivity, and measured.

The method according to the invention is suitable, for example, for use in a microwave sensor which is designed to determine the solids content of a flowable, aqueous medium.

Within the context of the invention, the reference is a mathematical variable which describes the basic contribution of each component to the relative determined permittivity. The reference may comprise a mathematical function of the relative permittivity or its real or imaginary part on the basis of frequency. The mathematical variable can also be a frequency-dependent vector.

Advantageous embodiments of the invention are the subject matter of the dependent claims.

One embodiment provides that only the real part of the relative permittivity is included when determining the lactose content a, protein content b and fat content c.

It has been found that the contributions of the individual lactose, protein and fat components are more apparent in the real part than in the imaginary part.

One embodiment provides that only the imaginary part of the relative permittivity is included when determining the lactose content a, protein content b and fat content c.

As an alternative to determining the proportions on the basis of the real part of the relative permittivity, the imaginary part of the relative permittivity can also be used to determine the individual lactose, protein and fat proportions.

One embodiment provides that the method comprises the method steps of:
measuring or providing the electrical conductivity of the medium,
compensating for the imaginary part of the relative permittivity on the basis of the measured or provided conductivity.

The imaginary part of the real permittivity describes a loss term that arises from constant repolarization of the molecules in the medium. This loss term is composed of polarization and conductivity losses. The conductivity losses are particularly evident at low frequencies and therefore, for media with a high conductivity (e.g., milk), conductivity compensation must be carried out for the imaginary part. The proportion of conductivity can be calculated using the following equation:

$${ε}_{r , L}^{″} = \frac{σ}{2 π f {ε}_{0}}$$

The electrical conductivity can be determined using a conductivity sensor. This can be part of the measuring assembly and thus integrated into the process line or can be designed as an external hand-held device with which the conductivity of a sample of the medium is or can be determined. Alternatively, the electrical conductivity of the medium can also be predefined by the user.

One embodiment provides that the milk is modeled as a four-component system in the calculation,
wherein the components comprise fat, lactose, proteins and water.

One embodiment provides that the water content of the medium is between 55, in particular 80, and 95 wt. %.

One embodiment provides that the method comprises the method step of:
specifying an operating point,
   wherein the operating point specifies a water content or fixed water content range,
   wherein at least the lactose reference, the protein reference and/or the fat reference differ for different operating points.

The operating point is specified by the user. The advantage of taking the expected water content into account is the higher degree of accuracy that can thus be achieved when determining the individual proportions. Within the context of the invention, an operating point comprises exactly one specific water content that is known to or assumed by the user, or a water content range that comprises a plurality of water contents.

One embodiment provides that the lactose reference, the protein reference and the fat reference are linearizable at the specified operating point.

One embodiment provides that the method comprises the method step of:
measuring the temperature of the medium,
   wherein the temperature of the medium is included when calculating the lactose content a, protein content b and fat content c and in particular when compensating for the imaginary part of the permittivity by means of the measured or provided conductivity.

In practice, the relative permittivity depends on the temperature and the measurement frequency. The desired degree of accuracy is achieved by a temperature measurement that takes into account the dependency of the medium properties on the temperature.

One embodiment provides that the lactose, protein and fat references are each temperature dependent.

One embodiment provides that the lactose reference comprises a frequency-dependent lactose vector \vec{l},
wherein the protein reference comprises a frequency-dependent protein vector \vec{p},
wherein the fat reference comprises a frequency-dependent fat vector \vec{f}.

One embodiment provides that the lactose content a, protein content b and fat content c are/can be determined by means of the equation

$$( \begin{matrix}a \\ b \\ c\end{matrix} ) = {A}^{- 1} \cdot ( \begin{matrix}\vec{M} ∘ {\vec{v}}_{1} \\ \vec{M} ∘ {\vec{v}}_{2} \\ \vec{M} ∘ {\vec{v}}_{3}\end{matrix} )$$

wherein the following applies for A

$$A = ( \begin{matrix}\vec{l} ∘ {\vec{v}}_{1} & \vec{p} ∘ {\vec{v}}_{1} & \vec{f} ∘ {\vec{v}}_{1} \\ \vec{l} ∘ {\vec{v}}_{2} & \vec{p} ∘ {\vec{v}}_{2} & \vec{f} ∘ {\vec{v}}_{2} \\ \vec{l} ∘ {\vec{v}}_{3} & \vec{p} ∘ {\vec{v}}_{3} & \vec{f} ∘ {\vec{v}}_{3}\end{matrix} )$$

wherein the basis vectors \vec{v}₁, \vec{v}₂ and \vec{v}₃ result from an orthogonalization process of the lactose vector \vec{l}, the protein vector \vec{p} and the fat vector \vec{f},
wherein \vec{M} represents the relative permittivity determined, for which it is assumed that \vec{M}=a·\vec{l}+b·\vec{p}+c·\vec{f}.

The matrix can comprise the vector products of the basis vectors and the corresponding component vector, or alternatively can already exist in the measuring assembly as a parameter matrix, i.e., with vector products already executed.

The basis vectors can also be stored as equations that describe the frequency-dependent behavior of the relative permittivity, or rather the real part or the imaginary part.

The measuring assembly according to the invention for determining a lactose content a, protein content b and fat content c of a flowable medium, in particular milk, a milk substitute and/or a milk product, comprises:
a measuring tube for conducting the medium;
at least one microwave antenna;
   wherein the at least one microwave antenna is arranged on the measuring tube;
transducer electronics designed to carry out the method according to the invention.

One embodiment provides that the measuring assembly comprises:
at least two microwave antennas,
   wherein the at least two microwave antennas are arranged on the measuring tube,
   wherein a first microwave antenna of the at least two microwave antennas has at least a first measuring range,
   wherein a second microwave antenna of the at least two microwave antennas has at least a second measuring range,
   wherein the first measuring range and the second measuring range collectively cover a frequency range of from 10 MHz to 50 GHz, in particular 50 MHz to 25 GHz and preferably 85 MHz to 14 GHz.

One embodiment provides that the transducer electronics comprises an electronic memory,
wherein the lactose vector \vec{l}, the protein vector \vec{p} and the fat vector \vec{f} are stored in the memory.

The invention is explained in greater detail with reference to the following figures, in which:

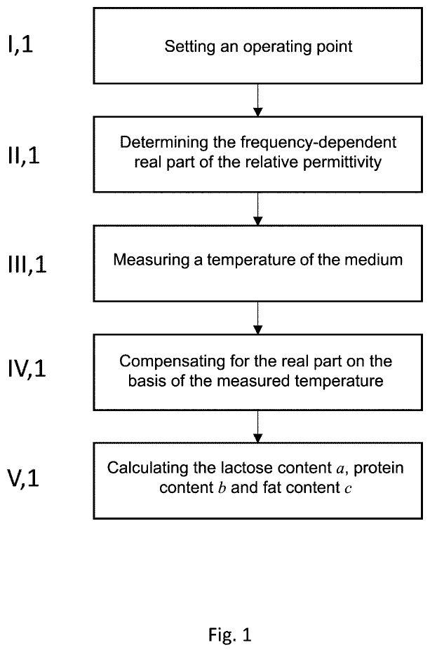

FIG. 1 shows a first embodiment of the method according to the invention;

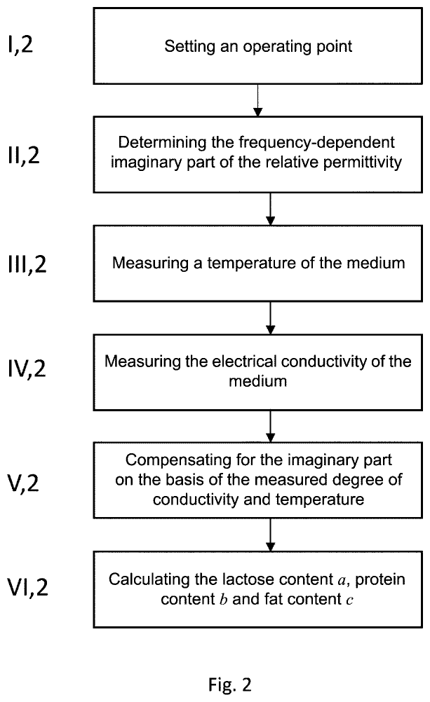

FIG. 2 shows a second embodiment of the method according to the invention;

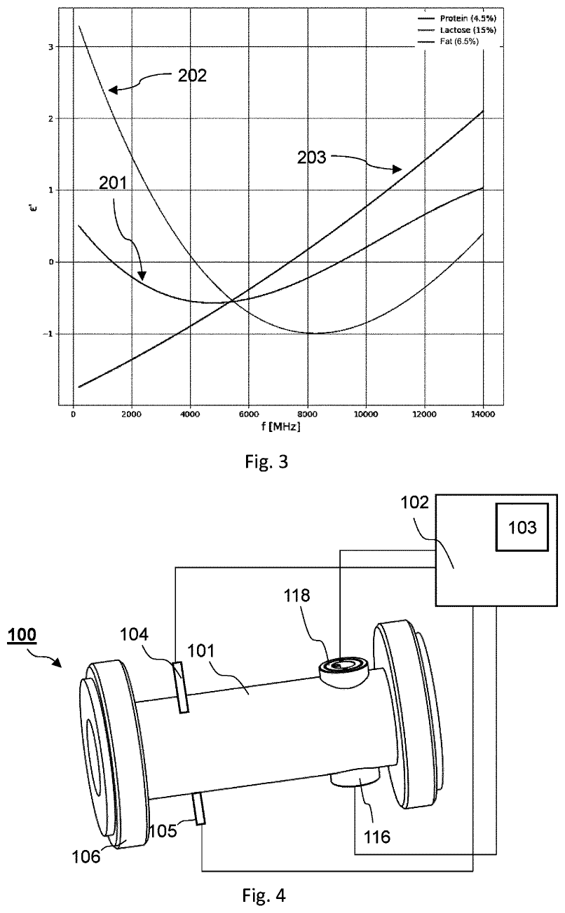

FIG. 3 shows the real part of the relative permittivity on the basis of frequency for a lactose reference, a protein reference and a fat reference; and

FIG. 4 shows an embodiment of the measuring assembly according to the invention.

FIG. 1 shows a first embodiment of the method according to the invention, which can or is to be carried out by means of a measuring assembly having a microwave antenna. In a first method step I,1, an operating point is specified. The operating point specifies the water content of the medium to be monitored or the water content range in which the water content expected for the medium lies. For milk, milk substitutes, milk products and milk substitute products, a water content of between 55, in particular 80, and 95 wt. % is assumed. The operating point can be specified by the operator of the measuring assembly on a corresponding display of the measuring assembly either on-site or via a central monitoring unit that is connected to the measuring assembly either contactlessly or via cables.

In a second method step II,1, the frequency-dependent relative permittivity of the medium is determined across a frequency range of a frequency spectrum. In the specific embodiment, the real part of the relative permittivity is determined and used to determine the individual proportions in the medium. The frequency spectrum has an upper limit of 50 GHz, in particular 25 GHz and preferably 14 GHz and a lower limit of 10 MHz, in particular 50 MHz and preferably 85 MHz. This is done using a microwave sensor. The microwave sensor is designed to send a microwave signal into the medium and, after interacting with the medium, to measure it again. The measured microwave signal is used to determine the real part of the relative permittivity for the frequency band of the microwave signal.

In a third method step III,1, the temperature of the medium is measured. For this purpose, a temperature sensor can be provided which is part of the measuring assembly. Alternatively, the temperature of the medium can be determined using a temperature sensor that is separate from the measuring assembly. In this case, the current temperature of the medium is provided to the transducer electronics.

In a fourth method step IV, 1, the determined real part of the relative permittivity is corrected or compensated for on the basis of the temperature measured. Alternatively, lactose, protein and/or fat references provided may be temperature dependent.

In a fifth method step V,1 the lactose content a, the protein content b and the fat content c are calculated on the basis of the real part determined and on the basis of a lactose reference, protein reference and fat reference that are provided. For modeling purposes, a four-component system is assumed for the milk, the milk substitute, the milk product and/or the milk substitute product. The components are fat, lactose, proteins and water. In order to achieve the highest possible degree of accuracy when determining the individual proportions, it is essential that the lactose reference, the protein reference and/or the fat reference is/are adapted to each operating point. This means that the lactose reference, the protein reference and the fat reference are linearizable at each operating point. According to the embodiment, the lactose reference is a frequency-dependent lactose vector \vec{l}. The same applies to the protein reference, which comprises a frequency-dependent protein vector \vec{p} and the fat reference, which accordingly comprises a frequency-dependent fat vector \vec{f}.

Using the lactose vector \vec{l}, the protein vector \vec{p} and the fat vector \vec{f}, the lactose content a, the protein content b and the fat content c can be determined, according to the following:

$$( \begin{matrix}a \\ b \\ c\end{matrix} ) = {A}^{- 1} \cdot ( \begin{matrix}\vec{M} ∘ {\vec{v}}_{1} \\ \vec{M} ∘ {\vec{v}}_{2} \\ \vec{M} ∘ {\vec{v}}_{3}\end{matrix} )$$

The following applies forA

$$A = ( \begin{matrix}\vec{l} ∘ {\vec{v}}_{1} & \vec{p} ∘ {\vec{v}}_{1} & \vec{f} ∘ {\vec{v}}_{1} \\ \vec{l} ∘ {\vec{v}}_{2} & \vec{p} ∘ {\vec{v}}_{2} & \vec{f} ∘ {\vec{v}}_{2} \\ \vec{l} ∘ {\vec{v}}_{3} & \vec{p} ∘ {\vec{v}}_{3} & \vec{f} ∘ {\vec{v}}_{3}\end{matrix} )$$

with the basis vectors \vec{v}₁, \vec{v}₂ and \vec{v}₃ which result from an orthogonalization process (e.g., Gram-Schmidt orthogonalization process) of the lactose vector \vec{l}, the protein vector \vec{p} and the fat vector \vec{f}. The matrix A is used to transform the vectors \vec{l}, \vec{p} and \vec{f} into the orthogonal system. The vector \vec{M} represents the relative permittivity determined, for which it is assumed that \vec{M}=a·\vec{l}+b·\vec{p}+c·\vec{f} applies.

Alternatively, the individual references can also be stored as mathematical functions.

FIG. 2 shows a second embodiment of the method according to the invention, which can or is to be carried out by means of a measuring assembly having a microwave antenna. An operating point is specified in a first method step I,2. The operating point specifies the water content of the medium to be monitored or the water content range in which the water content expected for the medium lies. The operating point can be specified by the operator of the measuring assembly on a corresponding display of the measuring assembly either on-site or via a central monitoring unit that is connected to the measuring assembly either contactlessly or via cables.

In a second method step II,2, the frequency-dependent relative permittivity of the medium is determined across a frequency range of a frequency spectrum. In the specific embodiment, the imaginary part of the relative permittivity is determined and used to determine the individual proportions in the medium. The frequency spectrum has an upper limit of 50 GHz, in particular 25 GHz and preferably 14 GHz and a lower limit of 10 MHz, in particular 50 MHz and preferably 85 MHz. The imaginary part is determined using a microwave sensor. The microwave sensor is designed to send a microwave signal into the medium and, after interacting with the medium, to measure it again. The measured microwave signal is used to determine the imaginary part of the relative permittivity for the frequency band of the microwave signal.

In a third method step III,2, the temperature of the medium is measured. For this purpose, a temperature sensor can be provided which is part of the measuring assembly. Alternatively, the temperature of the medium can be determined using a temperature sensor that is separate from the measuring assembly. In this case, the current temperature of the medium is provided to the transducer electronics.

In a fourth method step VI,2, the electrical conductivity of the medium is measured. For this purpose, a conductivity sensor can be provided, which is part of the measuring assembly. Alternatively, the conductivity of the medium can be determined using a conductivity sensor that is separate from the measuring assembly. In this case, the current conductivity in the medium is provided to the transducer electronics.

In a fifth method step V,2, the determined imaginary part of the relative permittivity is corrected or compensated for on the basis of temperature and conductivity. Alternatively, the lactose, protein and/or fat references provided may be dependent on the temperature and/or conductivity.

In a sixth method step VI,2, the lactose content a, the protein content b and the fat content c are calculated on the basis of the imaginary part determined and on the basis of a lactose reference, protein reference and fat reference that are provided.

FIG. 3 shows the real part of the relative permittivity on the basis of frequency for a protein reference 201, a lactose reference 202 and a fat reference 203. The individual references are each the difference between two reference measurements of different reference media, which differ only in the water content and in one of the remaining three components (protein, lactose and fat). This also means that two components of the reference media are substantially identical.

For the protein reference 201, the lactose and fat contents of the two reference media are substantially identical. For the protein reference 201 shown, there is a protein difference of approximately 4.5 wt. % between the two reference media. The water content of the two reference media is between 87 and 92 wt. %. The curve for of the protein reference 201 is partially parabolic with the lowest point at approximately 5 GHz.

For the lactose reference 202, the protein and fat content of the two reference media are substantially identical. For the lactose reference 202 shown, there is a lactose difference of approximately 15 wt. % between the two reference media. The water content of the two reference media is between 87 and 92 wt. %. The curve for the lactose reference 202 is parabolic with the lowest point at approximately 8 GHz.

For the fat reference 203, the lactose and protein contents of the two reference media are substantially identical. For the fat reference 203 shown, there is a fat difference of approximately 6.5 wt. % between the two reference media. The water content of the two reference media is between 80 and 86 wt. %. The curve for the fat reference 203 is substantially linear in some portions.

FIG. 4 shows an embodiment of the measuring assembly 100 according to the invention for determining a lactose content a, protein content b and fat content c of a flowable medium, in particular milk, a milk substitute and/or a milk product. The measuring assembly 100 comprises a measuring tube 101 for carrying the medium and two microwave antennas 116, 118 arranged opposite one another. The microwave antenna 116 is configured to feed a microwave signal into the medium when the medium is present in the measuring tube. The microwave signal covers a frequency range of from 10 MHz to 50 GHz, in particular 50 MHz to 25 GHz and preferably 85 MHz to 14 GHz. Alternatively, an additional pair of microwave antennas can also be provided. In this case, the two measuring ranges of the two pairs of microwave antenna collectively cover the frequency range of from 10 MHz to 50 GHz, in particular 50 MHz to 25 GHz and preferably 85 MHz to 14 GHz.

The microwave antenna 118 is configured to measure the microwave signal transmitted into the medium by the microwave antenna 116. The measured microwave signal is provided to transducer electronics 102, which is configured to carry out the method according to the invention. The transducer electronics 102 may be mechanically connected to the microwave antenna. If the individual components are determined on-site in the transducer electronics 102, the transducer electronics 102 has an electronic memory 103 in which the fat references are stored as a frequency-dependent fat vector \vec{f}. Alternatively, the individual components can be determined in a master computing unit that communicates with the transducer electronics 102 via cables or wirelessly.

In addition to the microwave antennas, the measuring assembly has a temperature sensor 104, which is arranged in a lateral opening of the measuring tube 101 and positioned such that it is in contact with the medium when the medium is present. Alternatively, the temperature of the medium can also be determined using a temperature sensor that is arranged on the outer lateral surface of the measuring tube and is not in contact with the medium. The temperature sensor 104 is electrically connected to the transducer electronics 102 and is configured to provide current temperature measured values to the transducer electronics 102.

Furthermore, the measuring assembly 100 comprises a conductivity sensor 105 which, similarly to the temperature sensor 104, is arranged in a lateral opening of the measuring tube 101 and is designed to determine the electrical conductivity of the medium. The conductivity sensor 105 can be designed either such that it comes into contact with the medium or does not come into contact with the medium. The conductivity sensor 105 is electrically connected to the transducer electronics 102 and is configured to provide the transducer electronics 102 with measured electrical conductivity values.

The previous description of the illustrated embodiment refers to a transmission measurement method in which the microwave signal is generated by a microwave antenna and measured by another microwave antenna, usually positioned opposite thereto. Alternatively, the illustrated embodiment can also be operated in reflection mode. In this case, the first microwave antenna 116 is configured to radiate a microwave signal into the medium and simultaneously measure the microwave signal interacting with the medium. The second microwave antenna 118 is also configured to radiate a microwave signal into the medium and to measure the microwave signal interacting with the medium. In this case, the second microwave antenna 118 is not necessarily arranged opposite the first microwave antenna 116. The microwave signal generated by the first microwave antenna 116 covers a first measuring range, while the second microwave antenna 118 covers a second measuring range. The two measuring ranges collectively cover a frequency range of from 10 MHz to 50 GHz, in particular 50 MHz to 25 GHz and preferably 85 MHz to 14 GHz.

### LIST OF REFERENCE SIGNS

100 Measuring assembly
101 Measurement tube
102 Transducer electronics
103 Memory
104 Temperature sensor
105 Conductivity sensor
106 Process connection
116 First microwave antenna
118 Second microwave antenna
201 Protein Reference
202 Lactose Reference
203 Fat Reference

1-15. (canceled)

16. A method for determining a lactose content a, a protein content b, and a fat content c of a flowable medium, wherein the flowable medium is milk, a milk substitute, and/or a milk product, the method comprising:
determining a frequency-dependent relative permittivity of the flowable medium across a frequency range of a frequency spectrum;
wherein the frequency spectrum has an upper limit of 50 GHz, and
wherein the frequency spectrum has a lower limit of 10 MHz; and
calculating the lactose content a, the protein content b, and the fat content c based on the relative permittivity determined and based on a lactose reference, a protein reference, and a fat reference.

17. The method according to claim 16,
wherein only a real part of the relative permittivity is included when determining the lactose content a, the protein content b, and the fat content c.

18. The method according to claim 16,
wherein only an imaginary part of the relative permittivity is included when determining the lactose content a, the protein content b, and the fat content c.

19. The method according to claim 18, further comprising:
measuring or providing the electrical conductivity of the medium; and
compensating for the imaginary part of the relative permittivity on the basis of the measured or provided electrical conductivity.

20. The method according to claim 16,
wherein the milk is modeled as a four-component system in the calculation, and the four components are fat, lactose, proteins, and water.

21. The method according to claim 20,
wherein the water content of the medium is between 55 and 95 wt. %.

22. The method according to claim 16, further comprising:
specifying an operating point,
wherein the operating point specifies a water content or fixed water content range, and
wherein at least the lactose reference, the protein reference, and/or the fat reference differ for different operating points.

23. The method according to claim 22,
wherein the lactose reference, the protein reference, and the fat reference are linearizable at the specified operating point.

24. The method according to claim 19, further comprising:
measuring a temperature of the medium,
wherein the temperature of the medium is included when calculating the lactose content a, protein content b, and fat content c and when compensating for the imaginary part of the permittivity by means of the measured or determined conductivity.

25. The method according to claim 24,
wherein the lactose, protein, and fat references are each temperature dependent.

26. The method according to claim 16,
wherein the lactose reference comprises a frequency-dependent lactose vector \vec{l},
wherein the protein reference comprises a frequency-dependent protein vector \vec{p}, and
wherein the fat reference comprises a frequency-dependent fat vector \vec{f}.

27. The method according to claim 26,
wherein the lactose content a, protein content b, and fat content c are/can be determined by means of the equation
$$( \begin{matrix}a \\ b \\ c\end{matrix} ) = {A}^{- 1} \cdot ( \begin{matrix}\vec{M} ∘ {\vec{v}}_{1} \\ \vec{M} ∘ {\vec{v}}_{2} \\ \vec{M} ∘ {\vec{v}}_{3}\end{matrix} )$$
wherein the following applies for A
$$A = ( \begin{matrix}\vec{l} ∘ {\vec{v}}_{1} & \vec{p} ∘ {\vec{v}}_{1} & \vec{f} ∘ {\vec{v}}_{1} \\ \vec{l} ∘ {\vec{v}}_{2} & \vec{p} ∘ {\vec{v}}_{2} & \vec{f} ∘ {\vec{v}}_{2} \\ \vec{l} ∘ {\vec{v}}_{3} & \vec{p} ∘ {\vec{v}}_{3} & \vec{f} ∘ {\vec{v}}_{3}\end{matrix} )$$
wherein the basis vectors \vec{v}₁, \vec{v}₂ an \vec{v}₃ result from an orthogonalization process of the lactose vector \vec{l}, the protein vector \vec{p}, and the fat vector \vec{f}, and
wherein \vec{M} represents the relative permittivity determined, for which it is assumed that
$$\vec{M} = a \cdot \vec{l} + b \cdot \vec{p} + c \cdot \vec{f} .$$

28. A measuring assembly for determining a lactose content a, a protein content b, and a fat content c of a flowable medium, comprising:
a measuring tube for carrying the medium;
at least one microwave antenna, wherein the at least one microwave antenna is arranged on the measuring tube; and
transducer electronics that is designed to:
determine a frequency-dependent relative permittivity of the flowable medium across a frequency range of a frequency spectrum;
wherein the frequency spectrum has an upper limit of 50 GHz, and
wherein the frequency spectrum has a lower limit of 10 MHz; and
calculate the lactose content a, the protein content b, and the fat content c based on the relative permittivity determined and based on a lactose reference, a protein reference, and a fat reference.

29. The measuring assembly according to claim 28, further comprising:
at least two microwave antennas,
wherein the at least two microwave antennas are arranged on the measuring tube,
wherein a first microwave antenna of the at least two microwave antennas has at least a first measuring range,
wherein a second microwave antenna of the at least two microwave antennas has at least a second measuring range, and
wherein the first measuring range and the second measuring range collectively cover a frequency range of from 10 MHz to 50 GHz.

30. The measuring assembly according to claim 28,
wherein the transducer electronics includes an electronic memory, and
wherein the lactose vector \vec{l}, the protein vector \vec{p}, and the fat vector \vec{f} are stored in the electronic memory.
